(19) United States
(12) Patent Application Publication
(10) Pub. No.: US 2011/0274771 A1
(43) Pub. Date: Nov. 10, 2011

(54) Prenatal Syptomatic Kit

(76) Inventors: Jennifer M. Irizarry, Braintree, MA (US); Lisa Wu, South Easton, MA (US); Amanda Szczypien, Sutton, MA (US)

(21) Appl. No.: 12/775,963

(22) Filed: May 7, 2010

(51) Int. Cl. A61K 33/10; A61K 31/485; A61K 36/68; A61K 36/185; A61P 1/08; B65D 69/00; A61P 1/12; A61P 1/10; A61P 29/00; A61P 11/14; A61P 11/02; A61K 31/4402; A61P 1/04

(52) U.S. Cl. 424/687; 514/357; 514/289; 424/738; 424/769; 206/570

(57) ABSTRACT

A medication kit includes a plurality of individual non-prophylactic, non-urgent care symptomatic treatments configured to treat the symptoms of a pre-existing medical condition such as pregnancy. The treatments include a combination of pharmaceutical treatments and non-pharmaceutical treatments corresponding to particular symptoms often associated with the pre-existing condition. Each of the pharmaceutical treatments are configured for mutually independent dosage, and are predetermined by an authoritative organization such as the Food and Drug Administration to be safe for use at the mutually independent dosages, in combination with one another by otherwise healthy patients having the pre-existing medical condition, without expectation of adverse side effects.

### BACKGROUND

1. Technical Field

This invention relates to prearranged non-prophylactic, non-urgent care medical kits containing medications designed for the treatment of a particular preexisting condition. These kits may also include instructions for alternative treatments and related medical supplies to aid in the treatment of symptoms commonly associated with a pre-existing condition such as pregnancy.

2. Background Information

The usefulness of having a reserve of medical supplies readily available is well recognized. Consequently a wide variety of medical kits are available for use by non medical professionals. These include first aid and emergency kits as well as dispensing containers that may be used for drug storage. Although the idea of a portable collection of pharmaceuticals and medical supplies has been around for many years, they have remained largely focused on general preparedness rather than addressing the specialized needs of certain widely recognized pre-existing medical conditions.

Pre-existing medical conditions often present with an array of predictable symptomatic ailments. Many of these ailments may benefit from treatment using non prescription medicine available to the general public. Often, however, people without medical training seeking to treat their own symptoms are faced with a wide array of “over the counter” or non prescription medications to choose from to appropriately target their symptoms. They also face the concern that the use of multiple non prescription medications to treat a single or multiple symptoms may produce unwanted or potentially dangerous side effects. These concerns over properly targeted treatment and drug interactions are of particular concern to people already diagnosed with a medical condition that increases their risk such as high blood pressure or a pre-existing medical condition such as pregnancy. In 2004 alone, the National Center for Health Statistics estimates that there were 6.39 million pregnancies in the United States of America.

These concerns are often particularly acute when pregnancy is the pre-existing condition, as pregnant women often share a deep concern for the potential adverse effects medications may have on an unborn child. Many pregnant women also often do not have the time, energy or resources to seek the advice of a pharmacists or physicians. A woman may actually increase her risk by asking simple questions of under qualified personnel that give her the wrong information because they lack the experience, training, or resources to accurately diagnose her condition. The internet is considered a fast alternative for answers to medical questions, however this information is not always reliable or accurate and the average person may not be able to differentiate between a reliable source and an unreliable one.

Moreover, it is noted that conventional medical kits tend to be primarily designed for preventative or urgent care rather than symptomatic care targeted towards a particular medical condition. Other medical kits that may be designed for general purpose uses, may be overly complex, and thus prone to improper and potentially unsafe use, particularly by those with varying or rapidly progressing pre-existing conditions such as pregnancy. A need therefore exists for a medical kit specifically tailored for symptoms associated with pregnancy, that addresses the foregoing concerns.

### SUMMARY

One aspect of the present invention includes a prenatal medication kit including a container, and a plurality of individual non-prophylactic, non-urgent care symptomatic treatments configured for pregnant patients, and disposed within the container. The treatments include a combination of pharmaceutical treatments and non-pharmaceutical treatments corresponding to particular symptoms often occurring during pregnancy. Each of the pharmaceutical treatments are configured for mutually independent dosage, and are predetermined by at least one of the U.S. Food and Drug Administration (FDA) and the American Medical Association (AMA) to be safe for use in combination with one another, at their mutually independent dosages, by healthy, pregnant patients, during all three trimesters of pregnancy. The kit includes packaging configured to provide educational information including dosage and symptomatic information corresponding to each of the pharmaceutical and non-pharmaceutical treatments.

In another aspect of the invention, a medication kit includes a container and a plurality of individual non-prophylactic, non-urgent care symptomatic treatments configured to treat the symptoms of a pre-existing medical condition. The treatments include a combination of pharmaceutical treatments and non-pharmaceutical treatments corresponding to particular symptoms often associated with the pre-existing condition. Each of the pharmaceutical treatments are configured for mutually independent dosage, and are predetermined by an authoritative organization to be safe for use at the mutually independent dosages, in combination with one another by otherwise healthy patients having the pre-existing medical condition, without expectation of adverse side effects.

Still another aspect of the invention includes a method for delivering a plurality of non-prophylactic, non-urgent care symptomatic treatments to a patient having a pre-existing medical condition. The method includes disposing within a container, a plurality of individual non-prophylactic, non-urgent care symptomatic treatments configured to treat the symptoms of the pre-existing medical condition. The method also includes selecting the treatments to include a combination of pharmaceutical treatments and non-pharmaceutical treatments corresponding to particular symptoms associated with the pre-existing medical condition, and configuring the pharmaceutical treatments for mutually independent dosage. The pharmaceutical treatments are selected from among those having been predetermined by an authoritative organization to be safe for use at the mutually independent dosages, in combination with one another by otherwise healthy patients having the pre-existing medical condition, without expectation of adverse side effects. The patient is enabled to freely select from the plurality of symptomatic treatments.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a top view of one configuration of a kit embodiment of the present invention;

FIG. 2 is a top view of an alternative configuration of a kit embodiment of the present invention;

FIG. 3 is a top view of a further alternative configuration of a kit embodiment of the present invention.

### DETAILED DESCRIPTION

In the following detailed description, reference is made to the accompanying drawings that form a part hereof, and in which is shown by way of illustration, specific embodiments in which the invention may be practiced. These embodiments are described in sufficient detail to enable those skilled in the art to practice the invention, and it is to be understood that other embodiments may be utilized. It is also to be understood that structural, procedural and system changes may be made without departing from the spirit and scope of the present invention. In addition, well-known structures and techniques have not been shown in detail in order not to obscure the understanding of this description. The following detailed description is, therefore, not to be taken in a limiting sense, and the scope of the present invention is defined by the appended claims and their equivalents. For clarity of exposition, like features shown in the accompanying drawings are indicated with like reference numerals and similar features as shown in alternate embodiments in the drawings are indicated with similar reference numerals.

### General Overview

Embodiments of the present invention include a self contained kit allowing for the efficient self treatment of identified symptoms commonly occurring with a specific, pre-existing medical condition, such as pregnancy. These kits are configured to include basic instructions (such as FDA recommended dosages), and include contents (pharmaceuticals and/or medical supplies) that have been approved for use during all three trimesters, alone or in combination with one another, without the expectation of adverse interactions or side effects.

As used herein, the terms “freely used”, “safe”, “accepted”, “approved” and “recommended dosage” are used in the context of an authoritative organization to convey the use of pharmaceuticals (e.g., drugs), and non-pharmaceuticals (e.g., medical supplies and alternative therapies) without the expectation of adverse interaction or side effects by a person of average health. Authoritative organizations are those widely recognized in the art as representing professional opinion and analysis in the relevant field including, but not limited to, the American Medical Association (AMA), the Food and Drug Administration (FDA), Pharmaceutical Companies, and United States Pharmacopeia.

The terms “drug” and “pharmaceutical” are used interchangeably, to refer to substantially any chemical substance (e.g., medicine, or medication) taken or otherwise absorbed into the body to cure or ameliorate any symptoms of an illness or medical condition, whether available with or without a prescription from a professional medical care provider, and which are generally regulated by the FDA. The term “non-pharmaceutical” is used herein to denote substantially any materials, supplies, devices, or treatments used to cure or ameliorate symptoms of an illness, and which fall outside of the aforementioned definition of drug or pharmaceutical.

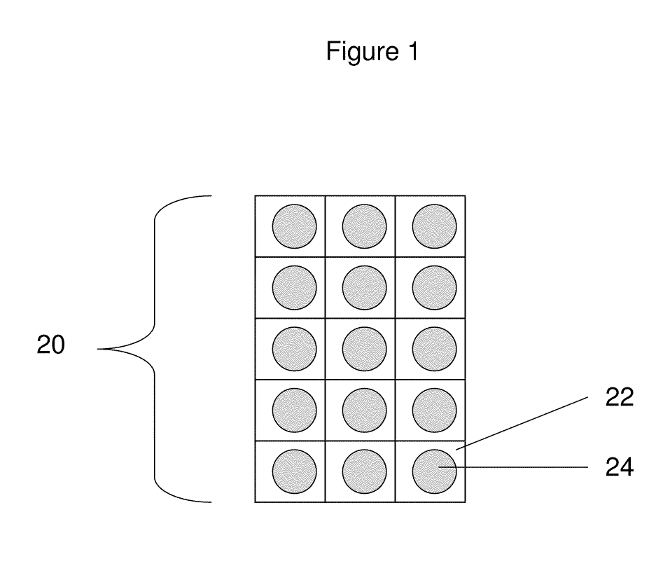

Referring to FIG. 1, an embodiment of the present invention includes a kit 20 including a container having a series of compartments 22 configured to receive individual non-prophylactic, non-urgent care symptomatic treatments, including pharmaceutical treatments (e.g., drugs) 24. The drugs 24 may be disposed in individual containers 24, and optionally, packaged in individual doses, as determined by one or more the above-referenced authoritative organizations. These drugs may include those that have been approved by one or more of the authoritative organizations to target specific symptoms commonly associated with a particular pre-existing condition such as pregnancy. Additionally, in particular embodiments, these selected drugs are chosen from “over the counter” or non prescription medications which may be replaced on an as-needed basis by the user. The drugs 24 are further selected to be safe for use by average users having the pre-existing medical condition, and to avoid negative side effects due to drug interaction with one another when taken together at each drug's recommended dosage, by the patient. Moreover, it is noted that each drug 24 is selected to have a single active ingredient, to avoid exposing the patient to drugs (such as those in combination remedies) that may be unnecessary in view of the patient's current symptoms. As mentioned above, in particular embodiments, the pre-existing condition is pregnancy and the drugs are pre-selected for the average user to include those non-prescription drugs that have been determined by authoritative organizations to be safe when used at recommended dosages, even when used in combination, during all three trimesters of pregnancy. These drugs are targeted to treat symptoms commonly associated with pregnancy. Some of these symptoms may include but are not limited to: runny nose, congestion, sneezing, sore throat, coughs, body aches, headaches, constipation, diarrhea, acid indigestion, nausea, and hemorrhoids. The use of drugs 24 having single active ingredients may be particularly useful in these embodiments to avoid exposing the mother's unborn child to drugs unnecessarily.

Particular embodiments tailored to pregnancy, may include from two to fifteen drugs (or otherwise biologically active compounds), selected from the following classes: antihistamines, nasal decongestants, cough suppressants, non-aspirin pain relievers, stool softeners or laxatives, dietary supplements, multivitamins, electrolyte replacements, anti-diarrhea medications, anti-gas medications, antacids, H2 blockers, topical astringents, non-steroidal anti-inflammatory drugs (NSAIDS), and saline sprays.

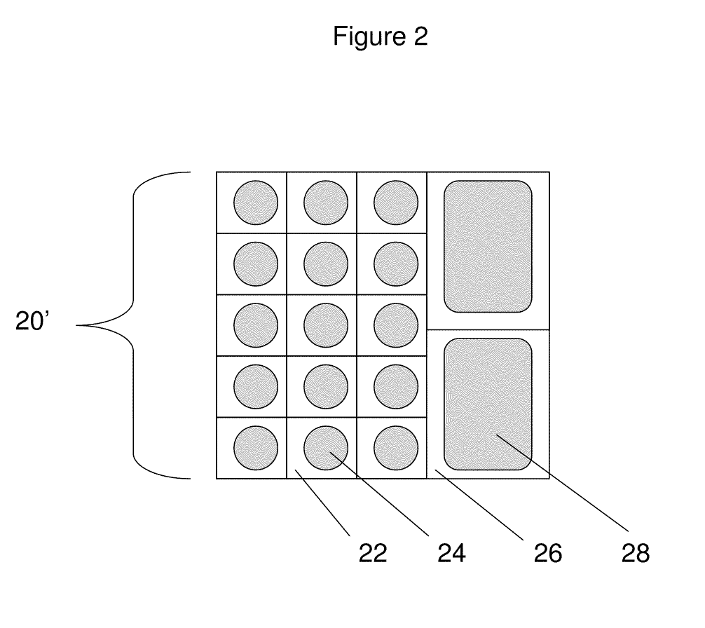

Referring now to FIG. 2, a further embodiment of the present invention is shown as kit 20′. This embodiment is substantially similar to the embodiment of FIG. 1, while also including a plurality of compartments 24 configured for holding non-pharmaceutical treatments, including medical supplies (and/or various alternative treatments) 28. In the particular embodiment shown, two storage compartments 24 are provided, although those skilled in the art will recognize that substantially any number of storage compartments 24 may be provided in substantially any configuration or shape. Compartments 24 may also be configured to hold multiple treatments, and/or supplies 28 of various types.

In particular embodiments, non-pharmaceutical treatments 28 are pre-selected for use in treating symptoms commonly associated with pregnancy. These treatments 28 may be chosen to relieve symptoms commonly associated with pregnancy such as runny nose, congestion, sneezing, sore throat, coughs, body aches, headaches, constipation, diarrhea, acid indigestion, nausea, and hemorrhoids. Examples of treatments 28 that may be selected include, but are not limited to ice-packs, heating pads, and nasal irrigation devices.

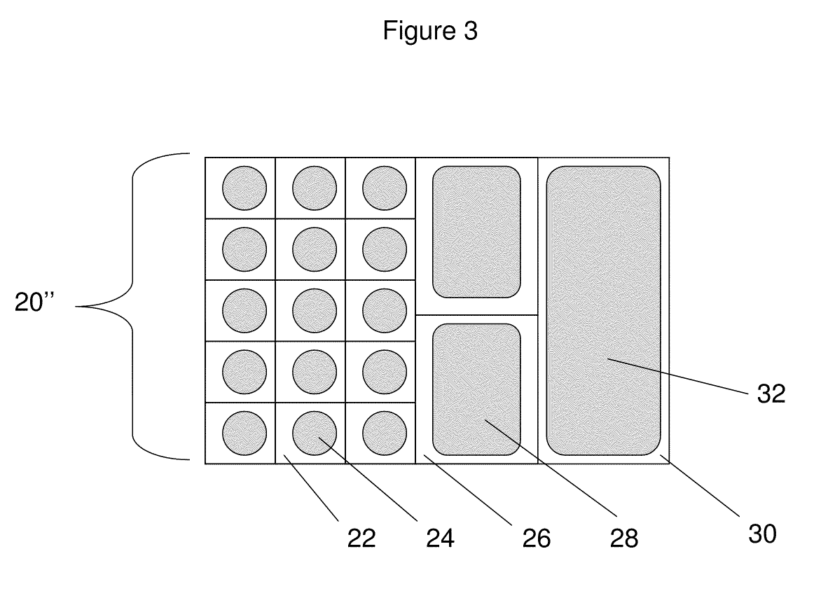

Referring now to FIG. 3, a further embodiment, shown as kit 20″, is substantially similar to the embodiment of FIG. 2 but includes the addition of one or more compartments 30 configured to contain information media 32 containing information related to the particular pre-existing condition. This information media may be configured to present the information in any one or more of a variety of formats including, but not limited to, written, audio, visual, and audio visual formats. For example, information media 32 may take the form of a written booklet having illustrations, charts, and tables. As another example, information media 32 may take the form of an audio recording in a digital or analog format. A further example may include a DVD (or Blu-Ray™ disc) or videocassette. Optionally, the media may be incorporated into the packaging of the kit, such as by printing onto the container or onto one or more labels affixed to the container.

The information recorded on media 32 may include, but is not limited to, lists of the drugs 24 and/or non-pharmaceutical supplies/alternative treatments 28 included in the kit 20, 20′, 20″ to address the particular pre-existing condition. This information may also include the class of such drugs and treatments, recommended dosages and other instructions for proper usage, the symptoms they address, active ingredients, and any drugs (e.g., which are not included in the kit, but which may otherwise be available to the patient) to avoid using in combination with those drugs/medications found in the kit. The information may also contain information specific to the particular pre-existing condition at issue (e.g., pregnancy), such as the trimester/week of pregnancy when particular drugs/medications (such as those not provided within the kit, but which may be available to the patient), etc., are considered generally safe for use, along with possible side effects and instructions on proper use. The informational material may also contain alternative therapies such as comfort or other non-pharmacological therapies.

It is noted that information pertaining to drug safety during particular trimesters may be provided. However, in particular embodiments, the kits of the present invention may be provided only with drugs/medications that have been approved for use during all three trimesters, and in combination with one another, to facilitate use and to help eliminate the possibility of user error. As a still further safeguard, the drugs 24 may be packaged into individual doses, to further simplify use of the kit and avoid errors by users who may have difficulty or may simply not take the time to read conventional drug labels.

As a variation of the foregoing embodiments, the structural container of the kit, as described but not limited to the figures provided, may be configured for re-use, e.g., for infant supplies after pregnancy. For example, the kit container may be configured to contain pediatric medications, medical supplies such as nasal irrigation devices, wipes, and diapers, etc.

The following illustrative example demonstrates certain aspects and embodiments of the present invention, and is not intended to limit the present invention to any one particular embodiment or set of features.

### Example

An example of an embodiment of the present invention is a pre-arranged kit designed for use by a woman who is experiencing various symptoms associated with, or otherwise commonly occurring during, pregnancy. Such a kit may contain drugs targeted towards relieving symptoms such as runny nose, congestion, sneezing, sore throat, coughs, body aches, headaches, constipation, diarrhea, acid indigestion, nausea, and hemorrhoids. The drugs are selected so they may be used in combination without the expectation of side effects. Individual drugs may be sub packaged into individual doses as an additional safeguard. Information listing the included drugs along with their active ingredients, symptoms they target, and medications to avoid while using a particular drug may be included. An example of a selection of drugs for a prenatal medical kit along with some of the relevant information is shown in the table below:

| | Therapeutic | Medications ok | Medications | | |
|---|---|---|---|---|---|
| DX/condition | class | to take | not to take | why | alternatives |
| symptoms: | | | | | |
| runny nose | antihistamine | chlorpheniramine | | 1st generation so sedating but | |
| | | | | used for many years safely; | |
| | | | | use over benadryl | |
| | | loratadine | | less sedating doesn't cross | saline nasal |
| | | | | placenta | spray |
| | | cromolyn nasal | | less systemic absorption | |
| | | spray | | longer to work | |
| | | benadry (not 1st | | drowsiness: used to aid sleep | |
| | | line) | | does cross | |
| congestion | decongestant | sudafed not 1st | | has ADR of gastroschisis. Not | mentholated |
| | | tri* | | for HBP!* | chest rub |
| | | oxymetazoline | | preferred over | |
| | | | | pseudoephedrine | |
| | | saline nasal spray | | b/c less absorption | |
| | | | phenylephrine | not as much data available for | hot shower |
| | | | | its use | |
| sneezing | antihistamine | loratadine | | | |
| | | benadryl (not 1st | | | |
| | | line) | | | |
| sore throat | | pectin cough | | | |
| | | drops | | | |
| | | sucrets contain | menthol or | crosses the placenta and risks | hard candy, |
| | | | phenol | unknown | |
| | | dextromethorphan | | considered safe but pectin is | lemon and |
| | | | | better alternative | honey |
| cough | expectorant | dextromethorphan | guaifenesin | link to neural tubual defects in | humidifier, |
| | cough supp. | | | 1st trimester | hydration, |
| | | | | | hot shower |
| | | | | | and liquids |
| body aches | pain reliever | acetaminophen | NSAIDs and | can cause bleeding risks and | hot/cold |
| | | | Aspirin | may increase miscarriage risk | compress |
| headache | pain reliever | acetaminophen | NSAIDs and | | topical creams |
| | | | Aspirin | | like camphor |
| constipation | stool softener | docusate sodium | | Studied in humans and shown | bisacodyl |
| | | | | not to have teratogenic effects. | |
| | | | | May cause hypomagnesaemia | |
| | | | | when used in high doses | |
| | | | | throughout the pregnancy | |
| | bulk forming | psyllium | | Generally considered safe to | |
| | laxative | | | use during pregnancy, | |
| | | | | however docusate sodium is | |
| | | | | preferred | |
| | stimulant | | senna | No data to show teratogenecity | |
| | laxative | | | in animals however there is no | |
| | | | | data available regarding its use | |
| | | | | in humans | |
| | stimulant | | bisacodyl | Limited data available, small | |
| | laxative | | | enough to pass placenta, but | |
| | | | | data suggests it may not. No | |
| | | | | reports of its use in pregnant | |
| | | | | women have been studied. | |
| | | | | May be alternative if other | |
| | | | | methods are ineffective. | |
| | hyperosmotic | | polyethylene | Large molecular weight | |
| | laxative | | glycol | unlikely to cross the placenta | |
| | | | | however there is a lack of | |
| | | | | human and animal data to | |
| | | | | substantiate its use compared | |
| | | | | to other options | |
| diarrhea | antidiarrhea | loperamide | | There is limited data available | fluid |
| | | | | to suggest loperamide would | replacement, |
| | | | | cause birth defects and animal | BRAT diet |
| | | | | data suggests the risk are low | |
| | salicylate | | bismuth | Use of bismuth subsalicylate in | |
| | | | subsalicylate | pregnancy have not been | |
| | | | | associated to teratogenecity | |
| | | | | however fetal risk cannot be | |
| | | | | ruled out due to known | |
| | | | | adverse events associated with | |
| | | | | use of other salicylates | |
| acid | antacid | calcium carbonate | | | avoid foods |
| indigestion | | | | | that trigger |
| | | | | | acid |
| | | | | | indigestion |
| | H₂ blocker | ranitidine | | does not have antiandrogenic | elevation of |
| | | | | effects of cimetidine so it is a | head during |
| | | | | safer alternative for long term | sleep |
| | | | | use in treatment of GERD | |
| | | | | however there is not data to | |
| | | | | suggest its use for short term | |
| | | | | treatment | |
| | H₂ blocker | | cimetidine | unlikely to cause fetal | |
| | | | | malformation, but animal | |
| | | | | studies have shown it has | |
| | | | | antiandrogenic effects. | |
| | | | | Alternative treatment options | |
| | | | | are recommended | |
| | H₂ blocker | | famotidine | known to cross the placenta, | |
| | | | | limited data is available | |
| | | | | regarding its safety in use | |
| | | | | during pregnancy. Safer | |
| | | | | alternatives are available | |
| | proton pump | | omeprazole | Unlikely to cause adverse | |
| | inhibitor | | | effects in fetus however lack of | |
| | | | | data is available to definitively | |
| | | | | say fetal defects were caused | |
| | | | | by other factors | |
| | adsorbant | Simethicone | | | |
| nausea/ | antihistamine | meclizine | | a known teratogen in animals | sea bands |
| vomiting | antihistamine | | | considered safe however has | ginger tabs |
| | | | | other adverse effects such as | |
| | | | | drowsiness | |
| | vitamin B6 | | | | |
| hemorrhoids | topical | witch hazel | | topically considered safe to | sitz bath |
| | astringent | | | use, it does not have systemic | |
| | | | | absorption | |
| | corticosteroid | | hydrocortisone | even with topical application, | |
| | | | cream | there is systemic absorption, | |
| | | | | corticosteroids are considered | |
| | | | | teratogenic even at low doses | |
| | | | | so their use should be very | |
| | | | | limited in the pregnant | |
| | | | | population | |

In addition the kit may contain alternative medical supplies such as an ice pack, heating pack and nasal irrigation device for a non-medicinal comfort regimen. Information may be included which discusses the drugs included in the kit (and optionally, drugs that may not be included in the kit). Information may also be provided regarding alternative, non-medicinal alternative treatments such as acupuncture, meditation, water immersion, etc., and relevant safety information. General health information regarding pregnancy may also be included which may anticipate questions and concerns such as what is normal to expect at different stages of pregnancy, and when it may be advisable to consult a doctor. The kit may be further configured to serve as a reusable container for infant medications, medical supplies and equipment after pregnancy.

In the preceding specification, the invention has been described with reference to specific exemplary embodiments for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

Having thus described the invention, what is claimed is:

1. A prenatal medication kit comprising:
a container;
a plurality of individual non-prophylactic, non-urgent care symptomatic treatments configured for pregnant patients, and disposed within said container;
said treatments including a combination of pharmaceutical treatments and non-pharmaceutical treatments corresponding to particular symptoms often occurring during pregnancy;
each of said pharmaceutical treatments being configured for mutually independent dosage;
each of said pharmaceutical treatments being predetermined by at least one of the U.S. Food and Drug Administration (FDA) and the American Medical Association (AMA) to be safe for use in combination with one another, at their mutually independent dosages, by healthy, pregnant patients, during all three trimesters of pregnancy; and
packaging for the kit, configured to provide educational information including dosage and symptomatic information corresponding to each of the pharmaceutical and non-pharmaceutical treatments.

2. A medication kit comprising:
a container;
a plurality of individual non-prophylactic, non-urgent care symptomatic treatments disposed within said container, and configured to treat the symptoms of a pre-existing medical condition;
said treatments including a combination of pharmaceutical treatments and non-pharmaceutical treatments corresponding to particular symptoms commonly associated with the pre-existing medical condition;
each of said pharmaceutical treatments being configured for mutually independent dosage; and
each of said pharmaceutical treatments being predetermined by an authoritative organization to be safe for use at said mutually independent dosages, in combination with one another by otherwise healthy patients having the pre-existing medical condition, without expectation of adverse side effects.

3. The medication kit of claim 2, wherein each of said pharmaceutical treatments are packaged in individual ones of said mutually independent dosages.

4. The medication kit of claim 2, wherein the authoritative organization is selected from the group consisting of the U.S. Food and Drug Administration (FDA), the American Medical Association (AMA), Pharmaceutical Companies, the United States Pharmacopeia™, and combinations thereof.

5. The medication kit according to claim 4 wherein:
said pre-existing medical condition is pregnancy and said pharmaceutical treatments are accepted by the authoritative organization for use in combination at recommended dosages in all three trimesters of the pregnancy.

6. The medication kit according to claim 5 wherein the pharmaceutical treatments comprise at least two drugs selected from the group consisting of antihistamines, nasal decongestants, cough suppressants, non-aspirin pain relievers, stool softeners or laxatives, dietary supplements, multivitamins, electrolyte replacements, anti-diarrhea medications, anti-gas medications, antacids, H2 blockers, topical astringents, non-steroidal anti-inflammatory drugs (NSAIDS), and saline sprays.

7. The medication kit according to claim 6 wherein the pharmaceutical treatments comprise up to fifteen drugs selected from the group consisting of antihistamines, nasal decongestants, cough suppressants, non-aspirin pain relievers, stool softeners or laxatives, dietary supplements, multivitamins, electrolyte replacements, anti-diarrhea medications, anti-gas medications, antacids, H2 blockers, topical astringents, NSAIDS, and saline sprays.

8. The medication kit of claim 7, wherein the pharmaceutical treatments are all available without a prescription.

9. The medication kit according to claim 2 wherein said non-pharmaceutical treatments comprise medical supplies.

10. The medication kit according to claim 9 wherein said non-pharmaceutical treatments comprise at least two items from the group consisting of an ice pack, heating pad, and a nasal irrigation device.

11. The medication kit according to claim 2, comprising media having medical information disposed thereon, directed towards the treatment of a pre-existing condition.

12. The medication kit according to claim 11 wherein said media is configured to provide educational information including dosage and symptomatic information corresponding to each of the pharmaceutical and non-pharmaceutical treatments.

13. The medication kit according to claim 12 wherein said media is configured to provide said educational information for alternative treatments which are absent from said kit but which may be otherwise available to a user.

14. The medication kit according to claim 13 wherein said media is configured to provide said educational information for proper usage of said alternative treatments.

15. The medication kit according to claim 14 wherein said information comprises descriptions of alternative non-pharmaceutical treatments for the pre-existing condition.

16. The medication kit according to claim 5 wherein said container is configured to be reusable as an infancy kit for storage of medical drugs and equipment appropriate for the infant care.

17. A method of delivering a plurality of non-prophylactic, non-urgent care symptomatic treatments to a patient having a pre-existing medical condition, the method comprising:
(a) providing a container;
(b) disposing within the container, a plurality of individual non-prophylactic, non-urgent care symptomatic treatments configured to treat the symptoms of the pre-existing medical condition;
(c) selecting the treatments to include a combination of pharmaceutical treatments and non-pharmaceutical treatments corresponding to particular symptoms associated with the pre-existing condition;
(d) configuring the pharmaceutical treatments for mutually independent dosage; and
(e) selecting the pharmaceutical treatments from among those having been predetermined by an authoritative organization to be safe for use at the mutually independent dosages, in combination with one another by otherwise healthy patients having the pre-existing medical condition, without expectation of adverse side effects; and
(f) enabling the patient to freely select from the plurality of symptomatic treatments.

18. The method of claim 17, comprising packaging each of said pharmaceutical treatments in individual ones of the mutually independent dosages.

19. The method of claim 17, wherein said selecting (e) further comprises selecting from among those pharmaceutical treatments that have been determined safe by an authoritative organization selected from the group consisting of the U.S. Food and Drug Administration (FDA), the American Medical Association (AMA), Pharmaceutical Companies, the United States Pharmacopeia™, and combinations thereof.

20. The method of claim 19 wherein said pre-existing medical condition is pregnancy and said selecting (e) further comprises selecting pharmaceutical treatments which have been accepted by the authoritative organization for use in combination at recommended dosages in all three trimesters of the pregnancy.

21. The method of claim 20 wherein said selecting (e) further comprises selecting at least two pharmaceutical treatments from the group consisting of antihistamines, nasal decongestants, cough suppressants, non-aspirin pain relievers, stool softeners or laxatives, dietary supplements, multivitamins, electrolyte replacements, anti-diarrhea medications, anti-gas medications, antacids, H2 blockers, topical astringents, non-steroidal anti-inflammatory drugs (NSAIDS), and saline sprays.

22. The method of claim 21 wherein said selecting (e) further comprises selecting up to fifteen pharmaceutical treatments from the group consisting of antihistamines, nasal decongestants, cough suppressants, non-aspirin pain relievers, stool softeners or laxatives, dietary supplements, multivitamins, electrolyte replacements, anti-diarrhea medications, anti-gas medications, antacids, H2 blockers, topical astringents, NSAIDS, and saline sprays.

23. The method of claim 22, wherein said selecting (e) further comprises selecting the pharmaceutical treatments from those available without a prescription.

24. The method of claim 17, wherein said selecting (c) comprises selecting non-pharmaceutical treatments in the form of medical supplies.

25. The method of claim 24 wherein said selecting (c) further comprises selecting at least two non-pharmaceutical treatments from the group consisting of an ice pack, heating pad, and a nasal irrigation device.

26. The method of claim 17, further comprising:
(g) disposing, within the container, media having medical information disposed thereon, directed towards the treatment of the pre-existing condition.

27. The method of claim 26, wherein said disposing (g) further comprises configuring the media to provide educational information including dosage and symptomatic information corresponding to each of the pharmaceutical and non-pharmaceutical treatments.

28. The method of claim 27, wherein said disposing (g) further comprises configuring the media to provide educational information for alternative treatments which are absent from said kit but which may be otherwise available to a user.

29. The method of claim 28, wherein said disposing (g) further comprises configuring the media to provide educational information for proper usage of the alternative treatments.

30. The method of claim 29, wherein said disposing (g) further comprises configuring the media to provide educational information including descriptions of alternative non-pharmaceutical treatments for the pre-existing condition.

31. The method of claim 20, comprising configuring the container to be reusable as an infancy kit for storage of medical drugs and equipment appropriate for the infant care.
